(19) United States
(12) Patent Application Publication
(10) Pub. No.: US 2018/0344987 A1
(43) Pub. Date: Dec. 6, 2018

(54) Splittable Expandable Braided Sheath

(71) Applicant: AngioDynamics, Inc., Latham, NY (US)

(72) Inventors: Carol Lancette, Fort Ann, NY (US); Theodore Beyer, Queensbury, NY (US); Teri Juckett, Queensbury, NY (US)

(73) Assignee: AngioDynamics, Inc., Latham, NY (US)

(21) Appl. No.: 15/995,971

(22) Filed: Jun. 1, 2018

Related U.S. Application Data
(60) Provisional application No. 62/513,506, filed on Jun. 1, 2017.

(51) Int. Cl. A61M 25/06

(52) U.S. Cl. CPC A61M 25/0668; A61M 2025/0681; A61M 2025/0675

(57) ABSTRACT

An introducer sheath device and method of use and removal for removing the introducer sheath device without having to remove a placed medical or surgical device are disclosed herein. The introducer sheath device includes an inner shaft, an outer shaft, a hub comprising a first hub portion, a second hub portion, a first thumb tab, a second thumb tab, a valve, and a tip portion.

### CROSS-REFERENCE TO RELATED APPLICATIONS

This application claims priority to and the benefit of U.S. Provisional Application 62/513,506, filed Jun. 1, 2017 and is hereby incorporated by reference.

### BACKGROUND

Surgical and/or medical devices are placed in several different ways, one such way being through an introducer sheath. A splittable introducer sheath is used to make removal of a medical device, such as a needle, dilator, or catheter easier and less disruptive to the patient. The splittable introducer sheath is capable of being split and removed without having to remove the inserted medical device from the patient.

Typically, prior art splittable introducer sheaths are formed by two pieces with two diametrically opposed score lines located on the sheath shaft. The two diametrically opposed score lines facilitate the splitting of the sheath when the user pulls on the sheath tabs in opposite directions, but the score lines also weaken the shaft structure, resulting in increased kinking potential and difficulty advancing the introducer sheath to the target site. Additionally, since the score lines are usually perforated, jagged edges are created along the score lines when the shafts are split and separate. These jagged edges could cause problems when the user is removing the introducer sheath from the target site.

There are numerous problems with the current splittable introducer sheaths known in the art. For example, current introducer sheaths can be too flexible, resulting in the sheaths becoming kinked, accordioned or even broken while accessing the vessel or organ to be treated. Moreover, upon being initially split, the splittable sheaths can break near the top of the sheath, making it difficult to fully peel the sheath away from the implanted medical device.

Therefore, there is a need in the art for an introducer sheath device that has a structure resulting in a more rigid sheath that is easily inserted into the vessel or organ of the patient.

The current invention has several advantages over the prior art. First, the current invention is comprised of an inner shaft structured to overlap with an outer shaft, resulting in a more rigid introducer sheath. The increased rigidity of the device's overlapping inner shaft and outer shaft structure reduces unwanted flexibility, increases kink resistance, and provides for more torqueability and control as compared to prior art introducer sheaths. The increased rigidity of the current device also makes the introducer sheath more stable when inserting into the patient and when co-axially inserting a medical device, such as a dilator or needle, through the sheath's lumen. In addition to the increased rigidity, the current invention is easier to split or separate, as the only portion of the device that the user has to split is the hub, rather than having to split down the entire prior art splittable sheath. The current invention also does not have the problems with jagged edges that plagued the prior art splittable introducer sheath.

### FIELD OF THE INVENTION

The present invention generally relates to vascular access devices. In particular, the invention is related to splittable introducer sheaths used to gain access to a vessel or other treatment site within a human body.

### SUMMARY OF THE DISCLOSURE

The present invention provides devices and methods for accessing a vessel and/or target area of a patient using an introducer sheath device, placing a medical device at the vessel and/or target area, and then removing the introducer sheath device from the medical device without having to remove the medical device from the vessel and/or target area of the patient. The introducer sheath device comprises an inner shaft and outer shaft. The inner shaft is coaxially situated within the outer shaft in an overlapping arrangement. The introducer sheath device further comprises a hub comprising a first hub portion, a second hub portion, and a valve. The hub is attached to the proximal end of the inner shaft and outer shaft.

### BRIEF DESCRIPTION OF THE DRAWINGS

FIG. 1A is an isometric view of the introducer sheath device.

FIG. 1B is an exploded isometric view of the introducer sheath device and the medical device to be introduced to the vessel or target site of the patient.

FIGS. 2A-2B show an isometric view of the inner shaft and inner shaft tab.

FIGS. 3A-3B show an isometric view of the outer shaft and outer shaft tab.

FIGS. 4-5 show partial isometric views of the inner shaft positioned within the outer shaft in an overlapping arrangement.

FIG. 6A shows a partial cross-section of the proximal portion of the introducer sheath device.

FIG. 6B shows a cross section of the inner shaft and outer shaft in an overlapping arrangement.

FIG. 7A shows a partial cross-section of an alternative embodiment of the proximal portion of the introducer sheath device of FIG. 6A.

FIG. 7B shows a cross-section of the inner shaft and outer shaft of the alternative embodiment of FIG. 6B.

FIGS. 8A-8C show a method of removing the introducer sheath device from the medical device.

### DETAILED DESCRIPTION OF THE FIGURES

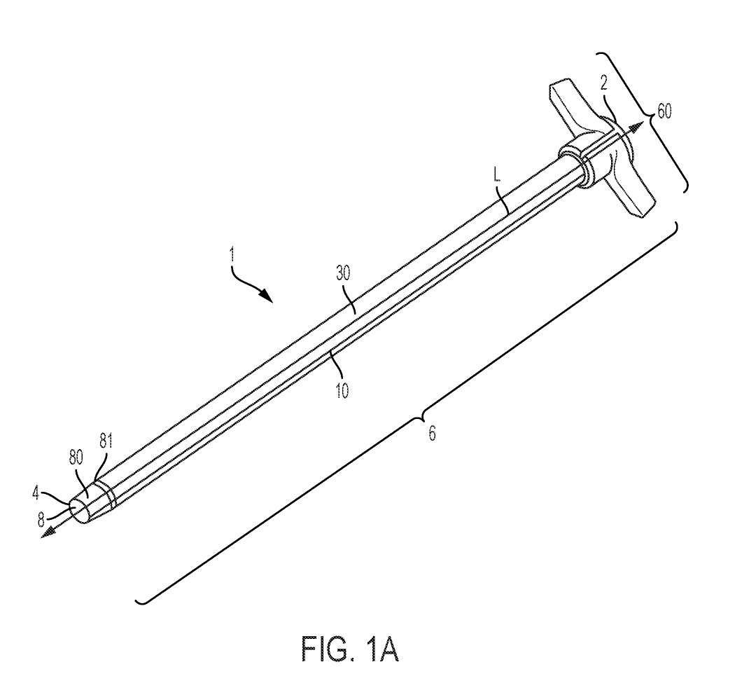

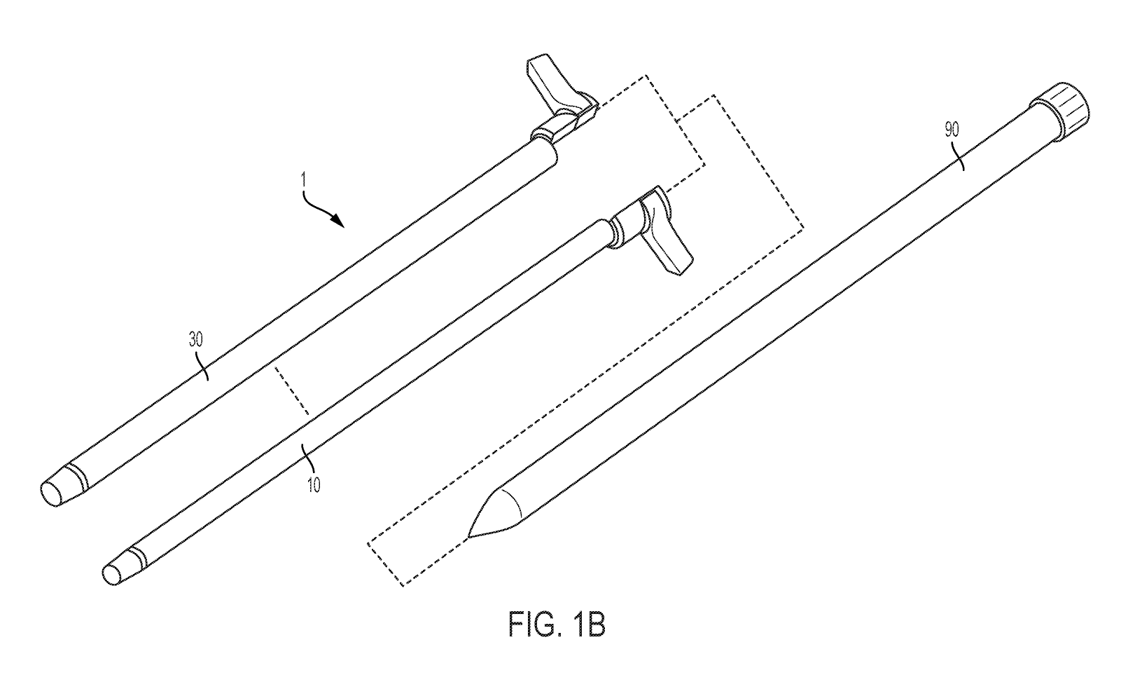

Referring now to FIGS. 1A-1B, a first embodiment of the introducer sheath device 1 is shown. The introducer sheath device 1 is comprised of an introducer sheath device proximal end 2, an introducer sheath device distal end 4, an introducer sheath device length 6 between the introducer sheath device proximal end 2 and the introducer sheath device distal end 4, an inner shaft 10, an outer shaft 30, a hub 60, and a tip portion 80. Also shown in FIG. 1A, the introducer sheath device comprises an introducer sheath device longitudinal axis L that extends along the introducer sheath device length. Also shown in FIG. 1B is a medical device 90, such as a dilator, needle, or catheter that may be coaxially inserted through an introducer sheath device lumen 8. As shown in FIG. 1B, the inner and outer shaft comprise two separate pieces that are split or have been separated and are connected to the first hub portion and second hub portion, respectively.

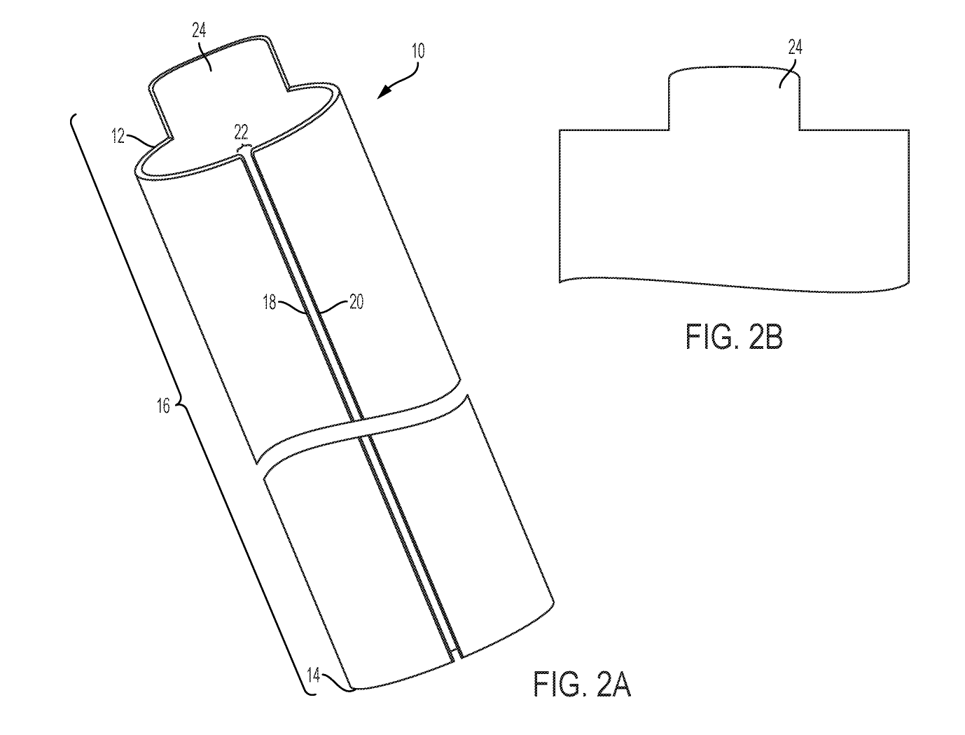

Referring to FIGS. 2A-2B, the inner shaft 10 of the introducer sheath device 1 comprises an inner shaft proximal end 12, an inner shaft distal end 14, an inner shaft length 16 extending between the inner shaft proximal end 12 and the inner shaft distal end 14, an inner shaft first edge 18, an inner shaft second edge 20, an inner shaft space 22 extending between the inner shaft first edge 18 and inner shaft second edge 20, an inner shaft tab 24, and an inner shaft reinforcement element (not shown). FIG. 2B is a partial view of the inner shaft in a two-dimensional, flattened perspective.

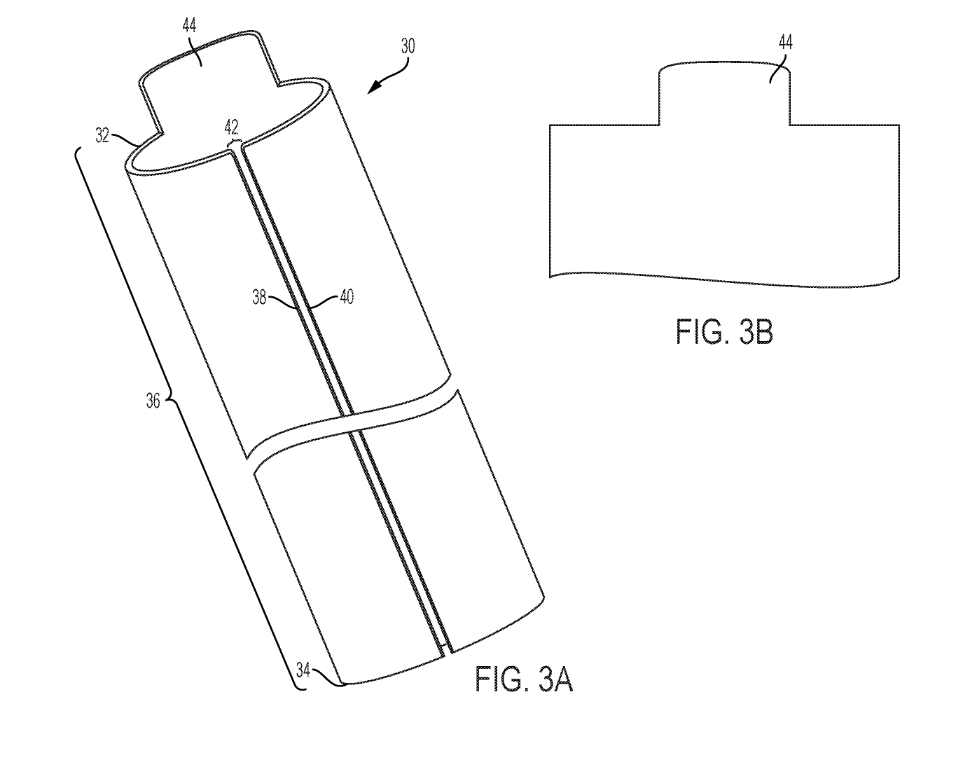

Referring to FIGS. 3A-3B, the outer shaft 30 of the introducer sheath device 1 comprises an outer shaft proximal end 32, an outer shaft distal end 34, an outer shaft length 36 that extends between the outer shaft proximal end 32 and the outer shaft distal end 34, an outer shaft first edge 38, an outer shaft second edge 40, an outer shaft space 42 extending between the outer shaft first edge 38 and outer shaft second edge 40, an outer shaft tab 44, and an outer shaft reinforcement element (not shown). FIG. 3B is a partial view of the outer shaft in a two-dimensional, flattened perspective.

The inner shaft 10 and/or outer shaft 30 may be made of the following materials including, but not limited to, poletetrafluoroethylene (PTFE), Teflon, high-density polyethylene (HDPE), or any other material known in the art. The introducer sheath device 1 may accommodate medical devices co-axially inserted through the introducer sheath device lumen 8 ranging in size from 5-12 French, and may have an inner diameter (ID) of ranging from 0.070″-0.162″ and an outer diameter (OD) ranging from 0.086″-0.178″. The inner diameter and outer diameter of the introducer sheath device 1 may be larger or smaller than the ranges expressed above and is not limited to the ranges above.

The inner shaft 10 and outer shaft 30 are formed by first co-extruding the inner shaft 10 and outer shaft 30 with an inner shaft reinforcement element (not shown) and outer shaft reinforcement element (not shown). The inner shaft 10 and outer shaft 30 are then cut to a desired length, creating an inner shaft proximal end 12 and outer shaft proximal end 32, an inner shaft distal end 14 and outer shaft distal end 34, and an inner shaft length 16 that extends between the inner shaft proximal end 12 and the inner shaft distal end 14 and an outer shaft length 36 that extends between the outer shaft proximal end 32 and outer shaft distal end 34. After the inner shaft 10 and outer shaft 30 are cut to the desired length, a tip portion 80 is then attached to the inner shaft distal end 14 and outer shaft distal end 34 via a tip portion proximal end 81. The tip portion 80 may be comprised of a single piece of material, including, but not limited to, nylon or another elastomeric material. The tip portion 80 can be made of a radiopaque material, a non-radiopaque material, or a hydrophilically coated material. The tip portion 80 may be attached to the inner shaft distal end 14 and outer shaft distal end 34 by any method known in the art, including, but not limited to heat welding.

Next, an inner shaft tab 24 and outer shaft tab 44 are formed along the inner shaft proximal end 12 and outer shaft proximal end 32. The inner shaft tab 24 may extend for a selected distance distal from the inner shaft proximal end 12 and the outer shaft tab 44 may extend for a selected distance distal from the outer shaft proximal end 32. One method of forming the inner shaft tab 24 and outer shaft tab 44 is by removing a selected amount of material of the inner shaft proximal end 12 and outer shaft proximal end 32 to form the tab profiles. After the inner shaft tab 24 and outer shaft tab 44 are formed, the inner shaft 10 and outer shaft 30 are cut along a longitudinal axis of the inner shaft length 16 and outer shaft length 36 and opposite of the inner shaft tab 24 and outer shaft tab 44. The cut made along the longitudinal axis of the inner shaft length 16 and outer shaft length 36 creates an inner shaft first edge 18 and outer shaft first edge 38, and an inner shaft second edge 20 and outer shaft second edge 40. The inner shaft first edge 18 and inner shaft second edge 20 extend along substantially the entire inner shaft length 16. The outer shaft first edge 38 and outer shaft second edge 40 extend substantially the entire outer shaft length 36. When the inner shaft 10 and outer shaft 30 are cut, the tip portion 80 is cut as well. Cutting the tip portion 80 aids in removal of the medical device 90 from the introducer sheath device 1. When the inner shaft 10 and outer shaft 30 are cut this results in an inner shaft space 22 between the inner shaft first edge 18 and inner shaft second edge 20, and an outer shaft space 42 between the outer shaft first edge 38 and the outer shaft second edge 40.

The inner shaft reinforcement element and outer shaft reinforcement element are typically made of a wire, but can also be any material known in the art that adds strength to the inner shaft 10 and/or outer shaft 30, including, but not limited to nylon, steel, nitinol, and any known polymer fibers. The inner shaft reinforcement element and outer shaft reinforcement element may be round, flat, or any other shape known in the art. The inner shaft reinforcement element and outer shaft reinforcement element may comprise any pattern known in the art that would add strength to the inner shaft 10 and outer shaft 30, including, but not limited to a braided pattern or a spiral pattern. The pitch of the pattern of the inner shaft reinforcement element and outer shaft reinforcement element may also be configured to make the inner shaft 10 and outer shaft 30 either more or less flexible, depending on the intended use of the introducer sheath device 1. If the pitch of the pattern of the inner shaft reinforcement element and outer shaft reinforcement element is tighter, the inner shaft 10 and outer shaft 30 will become less flexible. Conversely, if the pitch of the pattern of the inner shaft reinforcement element and outer shaft reinforcement element is looser, the inner shaft 10 and outer shaft 30 would be more flexible. The pitch of the pattern of the inner shaft reinforcement element and outer shaft reinforcement element may be customized depending on the place in the body that the user is trying to place the introducer sheath device 1. Harder to access areas would benefit from having a looser reinforcement element pattern, as the introducer sheath device 1 would be more flexible and be able to access these areas more easily. If the user is intending to place the introducer sheath device 1 in a straight vessel, a tighter reinforcement element pattern would be beneficial, as the introducer sheath device 1 may be more pushable. In order to reduce the occurrence of sharp edges resulting from the inner shaft reinforcement element and/or outer shaft reinforcement element when the inner shaft 10 and outer shaft 30 are cut, heat or any method known in the art can be applied to the inner shaft first edge 18, inner shaft second edge 20, outer shaft first edge 38, or outer shaft second edge 40.

In an alternate embodiment, a reinforcement element may only be used on one of either the inner shaft 10 or outer shaft 30, but not both shafts. The benefit of this embodiment is that the introducer sheath device 1 may be more flexible on one side than the other side and can be used to access harder to access target sites in the patient.

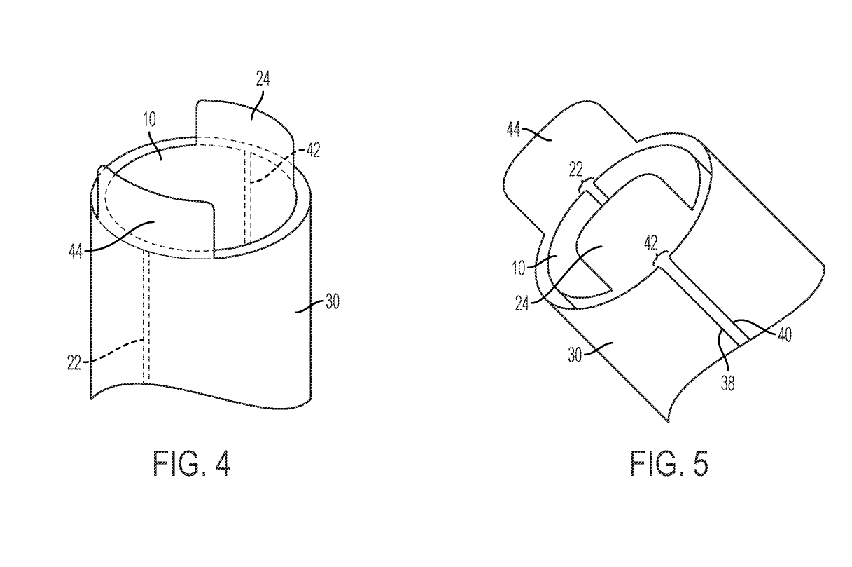

Referring now to FIGS. 4-5 the inner shaft 10 is positioned coaxially within the outer shaft 30 such that the outer shaft 30 overlaps the inner shaft 10 and the two shafts are held together by an interference fit. The inner shaft 10 and outer shaft 30 are arranged in an overlapping relationship such that the inner shaft tab 24 opposes the outer shaft tab 44. The inner shaft tab 24 may be positioned greater than or equal to 180 degrees or less than or equal to 180 degrees from the outer shaft tab 44. Additionally, the overlapping structure of the inner shaft 10 and outer shaft 30 result in the inner shaft space 22 opposing the outer shaft space 42. The inner shaft space 22 may be positioned greater than or equal to 180 degrees or less than or equal to 180 degrees from the outer shaft space 42. A key advantage to the introducer sheath device 1 is the overlapping structure and interference fit between the outer shaft 30 and the inner shaft 10, thus resulting in a more rigid and controllable device that is easier to remove from the medical device 90 than the prior art splittable introducer sheaths. Furthermore, the overlapping structure achieved via the interference fit of the outer shaft 30 and inner shaft 10 reduces potential likelihood of the user accidentally or unintentionally removing the inner and/or outer sheath from the medical device 90 being introduced to the patient.

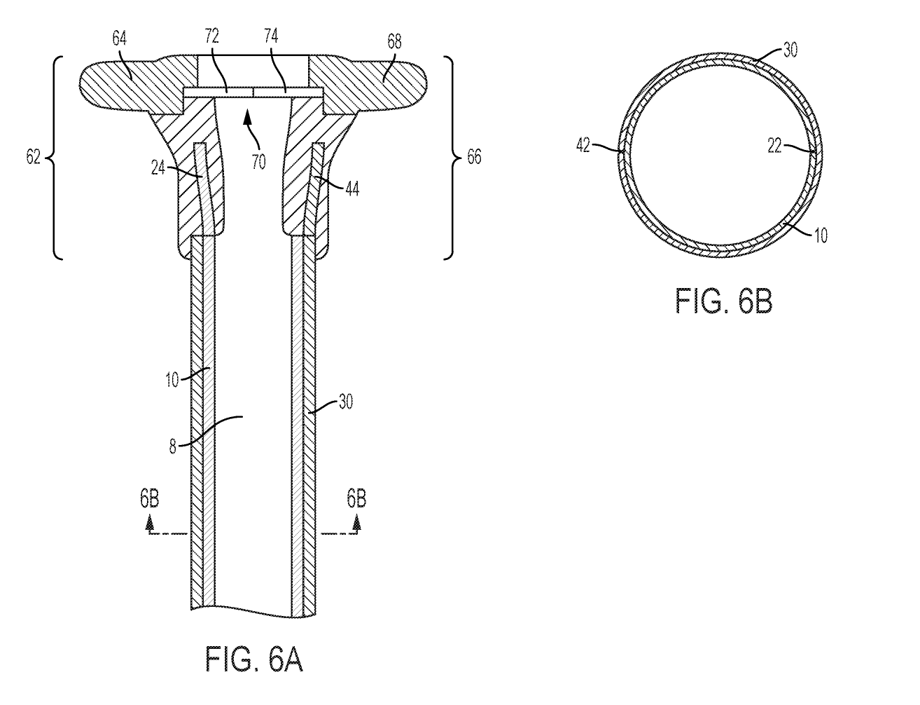

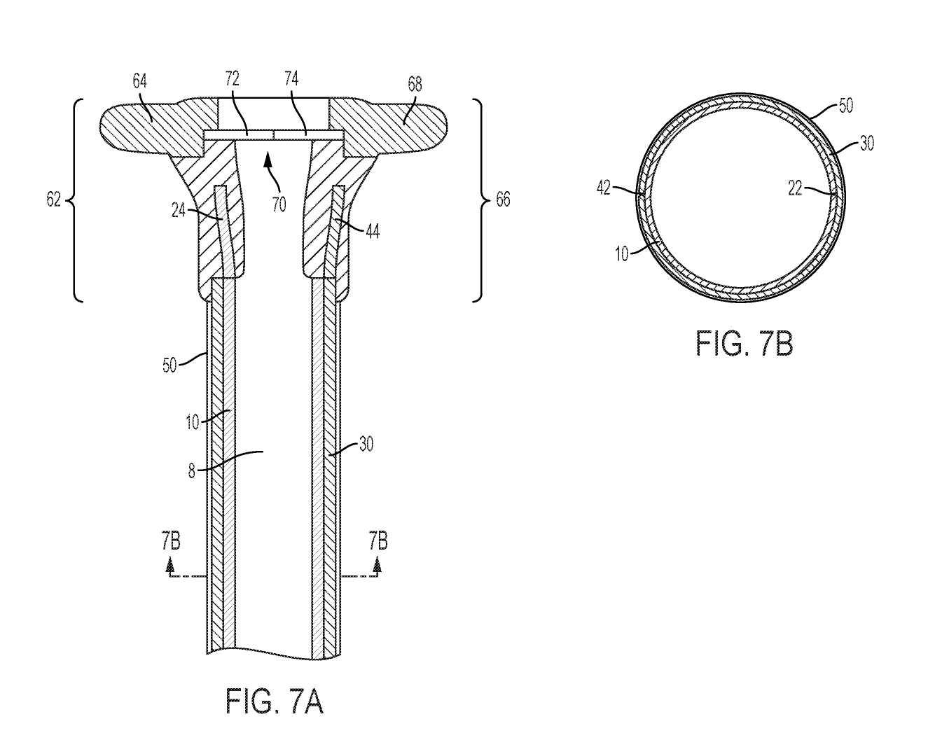

Referring now to FIG. 6A, the hub 60 of the introducer sheath device 1 is shown and is comprised of a first hub portion 62, a first thumb tab 64, a second hub portion 66, a second thumb tab 68, and a valve 70 comprising a first valve portion 72 and a second valve portion 74. The hub 60 can be made of plastic or any other material known in the art. The first hub portion 62 is comprised of a first thumb tab 64 connected to the first hub portion 62 and a first valve portion 72 seated within the first hub portion 62. The first thumb tab 64 may be molded and integral with the first hub portion 62 or alternatively the first thumb tab 64 may be molded separately and securely attached to the first hub portion 62 by means known in the art. The first valve portion 72 is seated within the first hub portion 62 by means of a compression fit. The second hub portion 66 is comprised of a second thumb tab 68 connected to the second hub portion 66 and a second valve portion 74 fit within the second hub portion 66. The second thumb tab 68 may be molded and integral with the second hub portion 66 or alternatively the second thumb tab 68 may be molded separately and securely attached to the second hub portion 66 by means known in the art. The second valve portion 74 is seated within the second hub portion 66 by means of a compression fit. Additionally, the hub 60 of the introducer sheath device 1 has a section of reduced material extending the length of the hub 60 and along the introducer sheath device longitudinal axis L. This section makes it easier to split the hub and ultimately, remove the introducer sheath device 1 from the medical device 90.

The first hub portion 62 is overmolded and thereby securely attached to the inner shaft tab 24. Similarly, the second hub portion 66 is overmolded and thereby securely attached to the outer shaft tab 44. There are several advantages of overmolding the first hub portion 62 so to be securely attached to the inner shaft tab 24 and overmolding the second hub portion 66 so to be securely attached to the outer shaft tab 44. For example, such a structure may add strength to the introducer sheath device 1; facilitate splitting of the introducer sheath device 1; make it easier to move the inner and outer shaft 30 relative to one another before removing the introducer sheath from the medical device 90; and facilitate in removal of the introducer sheath device 1 from the medical device 90. FIG. 6B shows a cross-sectional view of FIG. 6A in which the overlapping arrangement of the outer shaft 30 and inner shaft 10 is present. Additionally, the opposing arrangement of the inner shaft tab 24 and outer shaft tab 44 is shown.

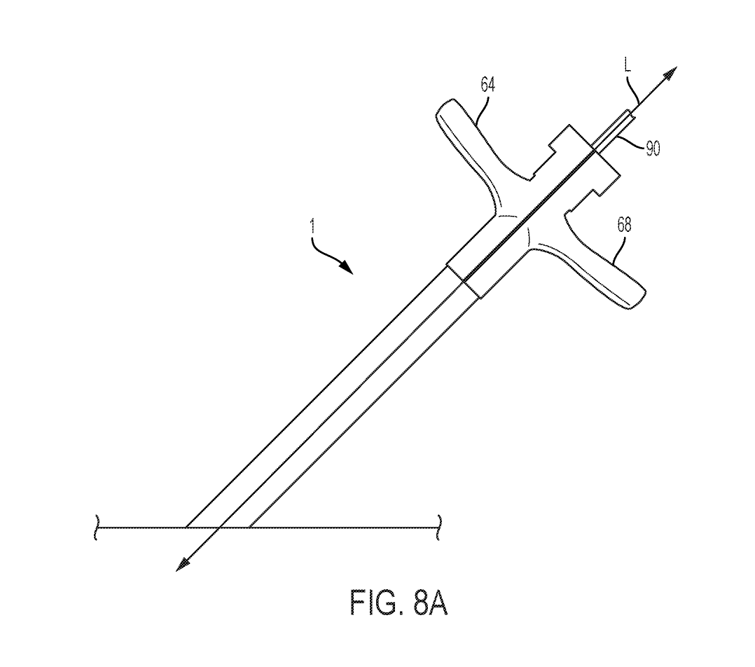

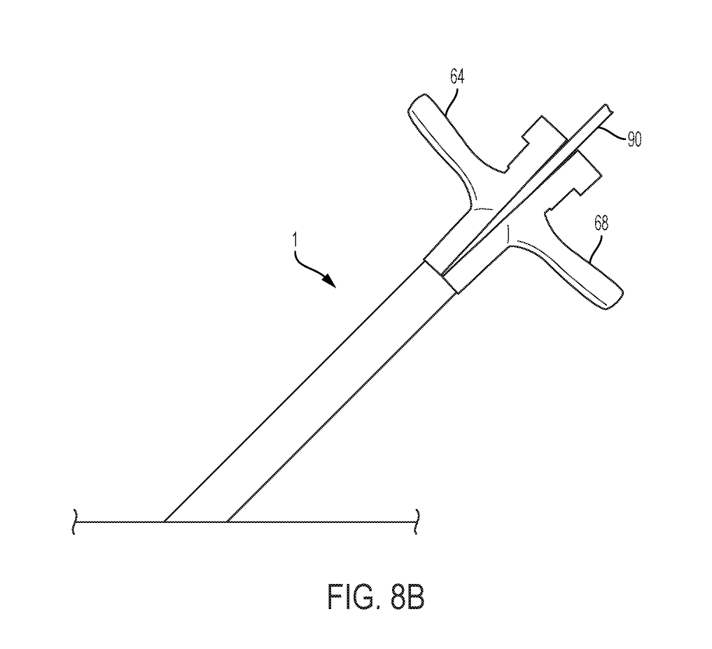

In an alternative embodiment shown in FIGS. 7A-7C, an outer layer 50 is applied after the inner sheath 10 and outer sheath 30 are created and fit in an overlapping arrangement. The outer layer 50 is comprised of an outer layer proximal end 52, an outer layer distal end, an outer layer length extending between the outer layer proximal end 52 and outer layer distal end, and an outer layer perforated section. The outer layer 50 can be formed of nylon or any other material known in the art. The outer layer length extends substantially along the outer layer length 56, surrounding the outer shaft 30 and/or inner shaft 10. The outer layer length 56 extends substantially along the outer shaft length 36 and inner shaft length 16. After the outer layer 50 is applied, an outer layer perforated section is made. The outer layer perforated section 58 is a section of thinner material to aid in the splitting of the introducer sheath device 1. The outer layer perforated section 58 extends in a straight line substantially along the outer layer length 56 and is made in line with the point where the first hub portion 62 and second hub portion 66 meet. The outer layer 50 can be hydrophilically coated to aid the insertion of the introducer sheath device 1 within the body. The outer layer perforated section 58 can also be made in a spiral shape, allowing the outer layer 50 to be unwound, rather than split down the middle and separated from the medical device 90 along the middle. FIG. 7B shows a cross-sectional view of FIG. 7A in which the overlapping arrangement of the outer shaft 30 and inner shaft 10 is present. Additionally, the opposing arrangement of the inner shaft tab 24 and outer shaft tab 44 is shown.

In another alternate embodiment (not shown), the inner shaft 10 and/or outer shaft 30 may include one or more rib(s) that extend substantially the entire inner shaft length 16 and/or outer shaft length 36. A stretchable material may be placed between each of the at least one rib(s). The at least one rib extending along substantially the entire inner shaft length 16 and/or outer shaft length 36 provides for added structure and rigidity. The stretchable material extending between the at least one rib(s) provides for added flexibility and supports expansion of the inner shaft 10 and/or outer shaft 30. One advantage of this embodiment is to provide the introducer sheath device 1 the ability to accommodate a range of different sized medical devices to be introduced using the sheath. The at least one rib(s) may be made of a variety of strengthening material including, but not limited to plastic, metal, etc.

A method of placing the introducer sheath device 1 of the current invention is described herein. First, a vessel or target site of a patient is accessed using a needle having a needle proximal end, a needle distal end, and an accessible lumen. Next, a guidewire having a guidewire distal end and a guidewire proximal end is placed through the lumen of the needle used to access the vessel or target site of the patient. The needle is then removed over the guidewire proximal end. The introducer sheath device 1 is then placed over the guidewire proximal end and advanced distally, providing the introducer sheath device 1 with access to the vessel or target site. Once the introducer sheath device 1 has accessed the vessel or target site of the patient, the guidewire is removed proximally through the introducer sheath device proximal end. After the introducer sheath device 1 is placed and the guidewire has been removed, a medical device 90 can be placed through the valve 70 and into the introducer sheath device lumen 8. The medical device 90 may include, but is not limited to, a dilator, catheter, or ablation device. In another embodiment, the medical device 90 may come preassembled within the introducer sheath device 1 in a form of a kit. The valve 70 of the introducer sheath device 1 prevents unwanted flow of blood and air through the introducer sheath device 1. As described above, one advantage for the overlapping configuration of the inner shaft 10 and outer shaft 30 of the introducer sheath device 1 is that the added rigidity aids in the pushability of the introducer sheath device, making it easier to access the vessel or target area of the patient.

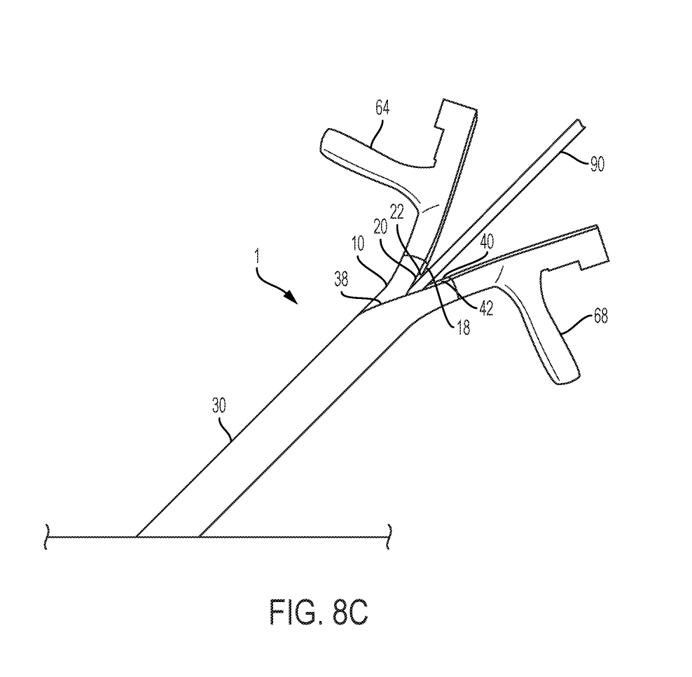

Now referring to FIGS. 8A-8C a method of removing the introducer sheath device 1 from the medical device 90 introduced to the vessel or target site of the patient is shown. The introducer sheath device 1 has a longitudinal axis L. First, the user splits the hub 60 by applying downward force to the first thumb tab 64 and second thumb tab 68 of the hub 60, thereby splitting (or cracking, opening, etc.) the hub along the hub 60. Splitting of the hub 60 results in the separation of the first hub portion 62 and the second hub portion 66. Next, the user pulls the first hub portion 62 and second hub portion 66 away from the introducer sheath device longitudinal axis L, exposing a portion of the medical device 90 in a proximal to distal direction. The first hub portion 62 and second hub portion 66 continue to be pulled apart, resulting in the outer shaft space 42 enlarging and the outer shaft 30 to be separated from the inner shaft 10 in a proximal to distal direction. After the outer shaft 30 has been separated from the inner shaft 10, the inner shaft 10 is pulled away from the introducer sheath device longitudinal axis L. This results in the inner shaft space 22 enlarging and the inner shaft 10 to separate from the medical device 90. Alternatively, after the hub has been split, the user may pull the first hub portion in a proximal direction and substantially along the introducer sheath device longitudinal axis, thereby removing the inner shaft. Next, the user may pull the second hub portion in a proximal direction and substantially along the introducer sheath device longitudinal axis, thereby removing the outer shaft.

1. A device comprising:
an inner shaft comprising an inner shaft proximal end, an inner shaft distal end, an inner shaft length between the inner shaft proximal end and the inner shaft distal end, and an inner shaft tab extending for a selected distance distal from the inner shaft proximal end;
an outer shaft comprising an outer shaft proximal end, an outer shaft distal end, an outer shaft length between the outer shaft proximal end and the outer shaft distal end, and an outer shaft tab extending for a selected distance distal from the outer shaft proximal end, the inner shaft oriented to be placed within the outer shaft;
a first hub portion comprising a first thumb tab and a first valve portion, the first hub portion connected to the inner shaft tab, the first valve portion connected to the first hub portion;
a second hub portion comprising a second thumb tab, and a second valve portion, the second hub portion connected to the outer shaft tab, the second valve portion connected to the second hub portion; and
a tip portion, the tip portion connected to the distal end of the outer layer.

2. The device of claim 1, wherein the inner shaft further comprises an inner shaft first edge extending along substantially the entire inner shaft length and an inner shaft second edge extending along substantially the entire inner shaft length.

3. The device of claim 1, wherein the outer shaft further comprises an outer shaft first edge extending along substantially the entire outer shaft length, an outer shaft second edge extending along substantially the entire outer shaft length.

4. The device of claim 1, wherein the inner shaft further comprises an inner shaft reinforcement element.

5. The device of claim 1, wherein the outer shaft further comprises an outer shaft reinforcement element.

6. The device of claim 1, further comprising an outer layer comprising an outer layer proximal end, an outer layer distal end, an outer layer length between the outer layer proximal end and the outer layer distal end, a perforated section extending along substantially the entire outer layer length, the outer layer surrounding at least a portion of the outer shaft and at least a portion of the inner shaft, the outer layer extending along substantially the entire inner shaft length and outer shaft length.

7. The device of claim 6, wherein the second hub portion abuts the proximal end of the outer layer.

8. The device of claim 6, wherein the first hub portion abuts the proximal end of the outer layer.

9. The device of claim 1, wherein the tip portion is comprised of a radiopaque material.

10. The device of claim 1, wherein the inner shaft tab is situated within the first hub portion.

11. The device of claim 1, wherein the outer shaft tab is situated within the second hub portion.
